(19) United States
(12) Patent Application Publication
(10) Pub. No.: US 2025/0215003 A1
(43) Pub. Date: Jul. 3, 2025

(54) AN IMPROVED PROCESS FOR THE PREPARATION OF RUXOLITINIB

(71) Applicant: GRANULES INDIA LIMITED, Hyderabad (IN)

(72) Inventors: Venkateswara Rao Kalapala, Hyderabad (IN); Srinivas Areveli, Hyderabad (IN); Veda Vyas Kokkiligadda, Hyderabad (IN); Sriram Rampalli, Hyderabad (IN); Sampath Kumar Upparapalli, Hyderabad (IN); Chennakesava Reddy Bandi, Hyderabad (IN); Basaveswara Rao Bavireddi, Hyderabad (IN)

(21) Appl. No.: 18/852,555

(22) PCT Filed: Aug. 21, 2023
(86) PCT No.: PCT/IB2023/058317

§ 371 Date: Sep. 30, 2024

(30) Foreign Application Priority Data
Aug. 22, 2022 (IN) 202241047750

(51) Int. Cl. C07D 487/04

(52) U.S. Cl. CPC C07D 487/04

(57) ABSTRACT

The main objective of the present invention is to improve the process for the preparation of ruxolitinib using protected (E)-N-(3-(Dimethylamino)-2-(7H-pyrrolo [2,3-d]pyrimidin-4-yl) allylidene)-N-methylmethanaminium chloride hydrochloride and (R)-5-cyclopentylpyrazolidin-3-one D-tartrate with higher yields on commercial scale. Formula (I):

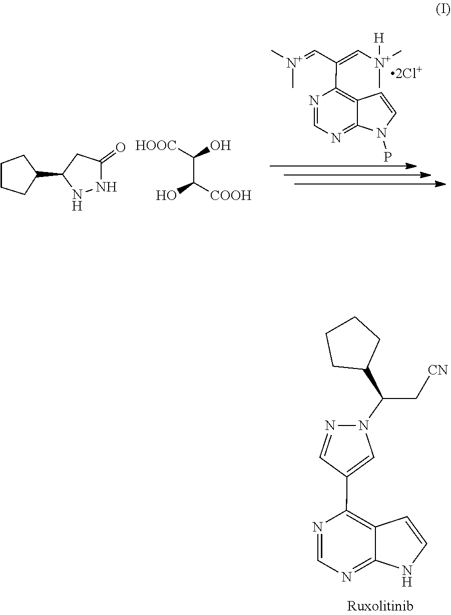

### FIELD OF THE INVENTION

The present application relates to improved methods for the preparation of ruxolitinib or pharmaceutically acceptable salt thereof, and methods for preparing related intermediates.

### BACKGROUND OF THE INVENTION

Ruxolitinib is a selective JAK1/JAK2 tyrosine kinase inhibitor developed by Incyte Corporation and Novartis Corporation. It is the first drug (Trade name: Jakafi) approved by the US FDA in November 2011 for the treatment of myelofibrosis, and indications thereof are intermediate or high-risk myelofibrosis, including primary myelofibrosis, secondary polycythemia myelofibrosis and post-essential thrombocythemia myelofibrosis.

Ruxolitinib phosphate chemically known as (R)-3-(4-(7H-pyrrolo[2,3-d] pyrimidin-4-yl)-1H-pyrazol-1-yl)-3-cyclopentylpropanenitrile phosphate and has the following structural formula:

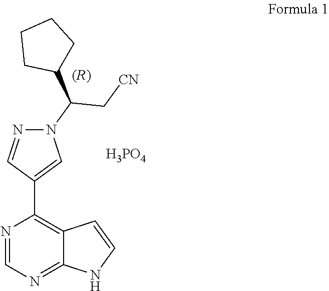

U.S. Pat. No. 7,598,257 discloses ruxolitinib, its pharmaceutically acceptable salts thereof. Also, it discloses a process for the preparation of Ruxolitinib which involves using HPLC methods to separate intermediates, also, involves in the use of column chromatography purification methods for the final product, which is laborious and time consuming and suffers from poor yield and low purity.

U.S. Pat. No. 8,410,265 discloses a process for the preparation of ruxolitinib, involves a chemical resolution of D-(+)-dibenzoyltartaric acid, asymmetric hydrogenation under the action of a chiral metal catalyst and a chiral small molecule catalyzed asymmetric synthesis route of the ruxolitinib. Wherein, the chemical resolution and repeated recrystallization are carried out, and the yield is low; the starting materials for asymmetric hydrogenation are not easy to prepare, and the chiral catalyst is difficult to prepare; the chiral small molecular catalyst is a non-commercial product, the preparation cost is high, and the stereoselectivity of the reaction is not high.

Above prior-art methods involve chiral resolution reactions at final stage which leads yield loss, chromatographic purification methods and the use of costly chiral and hazardous reagents. Using complex chiral agents increases the cost of production. Usage of chromatographic purification methods not suitable for industrial production.

In view of the above, still there is a great need to develop improved process methods for the preparation of ruxolitinib with high purity and suitable for industrial production. The inventors of the current invention focused more on resolving the shortcomings of the prior-art processes and developed a novel method for the preparation of ruxolitinib which is more useful for industrial production.

### SUMMARY OF THE INVENTION

In one aspect, the present invention provides a process for the preparation of ruxolitinib or a salt thereof, comprising:
reacting compound of formula 4 or a salt thereof,

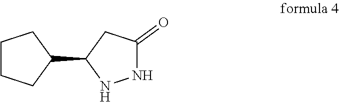 [SMILES: O=C1C[C@H](C2CCCC2)NN1]

with a compound of formula 5.

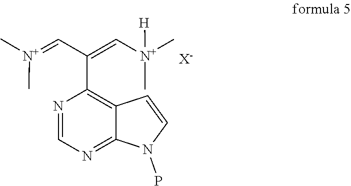

wherein, ‘P’ represents hydrogen or amine protecting group and X″ is a counter anion.

In another aspect, the present invention provides a process for the preparation of ruxolitinib or a salt thereof, comprising:
A) reacting compound of formula 4 or salt thereof,

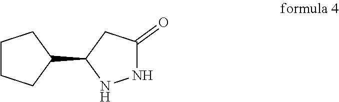 [SMILES: O=C1C[C@H](C2CCCC2)NN1]

with a compound of formula 5,

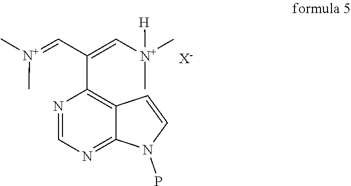

wherein, ‘P’ represents hydrogen or amine protecting group and X⁻ is a counter anion;
to provide acid compound of formula 6;

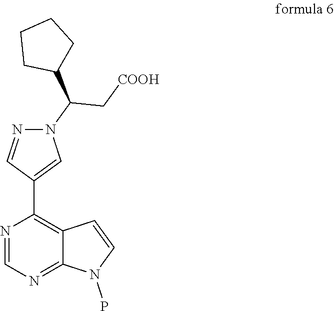

B) reacting the acid compound of formula 6 with alkyl chloroformates, dialkyl dicarbonates, alkyl or aryl sulphonyl chloride, sulfonic anhydrides to provide a compound of formula 7a;

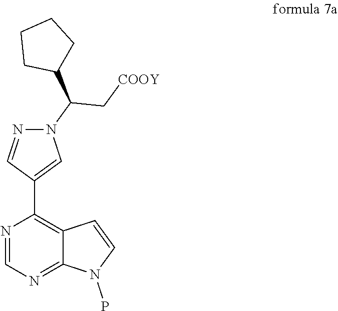

wherein ‘Y’ represents COOR, SO₂R and R is selected from alkyl, aryl or aralkyl group;
C) reacting the compound of formula 7a with an aminating agent to provide a compound of formula 8; and

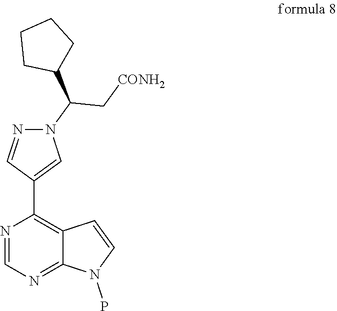

D) converting the compound of formula 8 into ruxolitinib or a salt thereof.

In another aspect, the present invention provides a process for the purification of compound of formula 6, comprising:

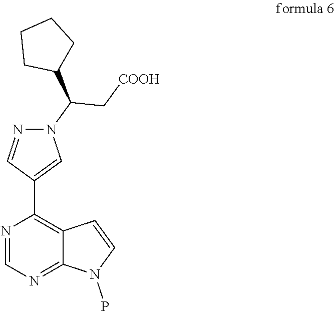

wherein, ‘P’ represents hydrogen or amine protecting group;
   reacting compound of formula 6 with dicyclohexylamine (DCHA) salt in a suitable solvent to provide dicyclohexylamine salt of compound of formula 6a, and

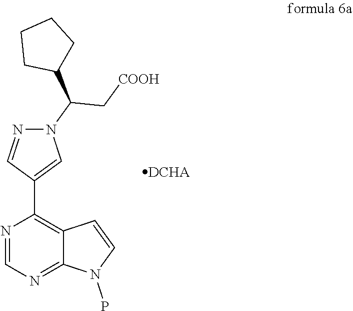

further converting dicyclohexylamine salt of compound of formula 6a into its free base by reacting it with base to provide pure compound of formula 6.

In another aspect, the present invention provides use of dicyclohexylamine salt of compound of formula 6a or pure compound of formula 6, in the preparation of ruxolitinib or a salt thereof.

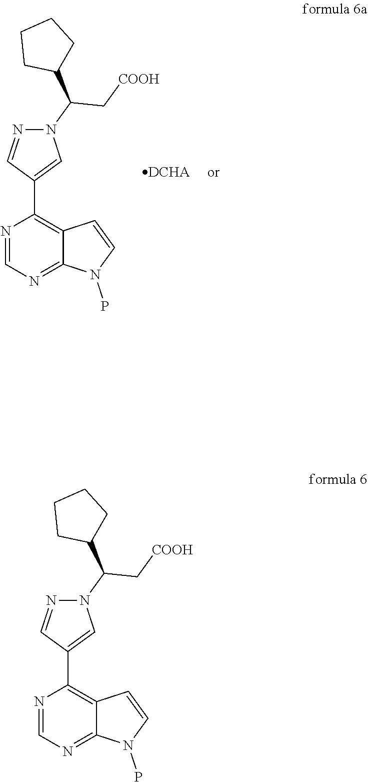

wherein, ‘P’ represents hydrogen or amine protecting group;

In another aspect, the present invention provides a process for the preparation of ruxolitinib or a salt thereof, comprising:
A) reacting the acid intermediate of formula 6,

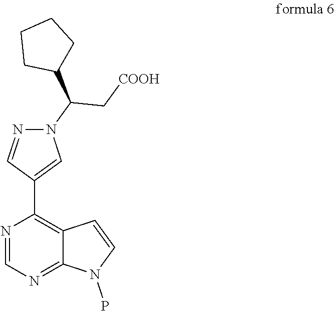

wherein, ‘P’ represents hydrogen or amine protecting group;
with alkyl chloroformates, dialkyl dicarbonates, alkyl or aryl sulphonyl chloride, sulfonic anhydrides to provide a compound of formula 7a;

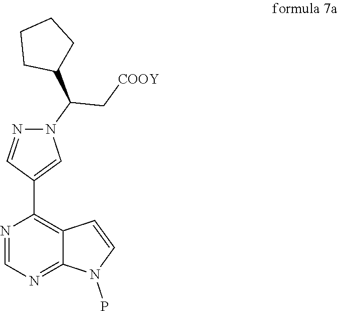

wherein ‘Y’ represents COOR, SO₂R and R is selected from alkyl, aryl or aralkyl group;
B) reacting the compound of formula 7a with an aminating agent to provide a compound of formula 8; and

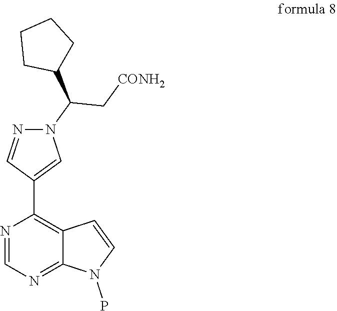

B) converting compound of formula 8 into ruxolitinib or a salt thereof.

### DETAILED DESCRIPTION

The “suitable solvent” used in the present invention can be selected from but not limited to “hydrocarbon solvents” such as n-pentane, n-hexane, n-heptane, cyclohexane, pet ether, benzene, toluene, xylene and the like; “ether solvents” such as dimethyl ether, diethyl ether, diisopropyl ether, methyl tert-butyl ether, 1,2-dimethoxyethane, tetrahydrofuran, 1,4-dioxane and the like; “ester solvents” such as methyl acetate, ethyl acetate, n-propyl acetate, isopropyl acetate, n-butyl acetate, isobutyl acetate, tert-butyl acetate and the like; “polar-aprotic solvents” such as dimethylacetamide, dimethylformamide, dimethyl sulfoxide, N-methylpyrrolidone and the like; “chloro solvents” such as dichloromethane, dichloroethane, chloroform, carbon tetrachloride and the like; “ketone solvents” such as acetone, methyl ethyl ketone, methyl isobutyl ketone and the like; “nitrile solvents” such as acetonitrile, propionitrile, isobutyronitrile and the like; “alcohol solvents” such as methanol, ethanol, n-propanol, iso-propanol, n-butanol, iso-butanol, 2-butanol, tert-butanol, ethane-1,2-diol, propane-1,2-diol and the like; “polar solvents” such as formic acid, acetic acid, and the like; and water or mixture of any of the afore mentioned solvents.

The suitable “amino protecting group ‘P’ can be selected from but not limited to benzyloxycarbonyl (Cbz), 2,2,2-trichloroethoxycarbonyl (Troc), 2-(trimethylsilyl) ethoxycarbonyl (Teoc), 2-(4-trifluoromethylphenylsulfonyl) ethoxycarbonyl (Tsc), tert-butoxycarbonyl (Boc), 1-adamantyloxycarbonyl (Adoc), 2-adamantylcarbonyl (2-Adoc), 2,4-dimethylpent-3-yloxycarbonyl (Doc), cyclohexyloxycarbonyl (Hoc), 1,1-dimethyl-2,2,2-trichloroethoxycarbonyl (TcBoc), vinyl, 2-chloroethyl, 2-phenylsulfonylethyl, p-nitrophenylsulfonyl, p-toluenesulfonyl, phenylsulfonyl, methanesulfonyl, allyl, benzyl, 2-nitrobenzyl, 4-nitrobenzyl, diphenyl-4-pyridylmethyl, N′,N′-dimethylhydrazino, methoxymethyl, tert-butoxymethyl (Bum), benzyloxymethyl (Bom), 2-tetrahydropyranyl (THP), tri (C1-4alkyl) silyl, 1,1-diethoxymethyl, 2-(trimethylsilyl) ethoxymethyl (SEM), N-pivaloyloxymethyl (POM) and the like.

The “suitable deprotecting agent” is selected based on the protecting group employed. The suitable deprotecting agent can be selected from but not limited to acids such as hydrochloric acid, hydrobromic acid, sulfuric acid, nitric acid, aq. phosphoric acid, trifluoroacetic acid, methane sulfonic acid, p-toluenesulfonic acid, trifluoroacetic anhydride, lithium tetrafluoroborate, boron trifluoride-diethyl etherate; acetyl chloride in combination with alcohols; bases such as alkali metal hydroxides, alkali metal carbonates, cesium carbonate/imidazole, alkali metal bicarbonates, ammonia, aqueous ammonia, ammonium cerium (IV) nitrate (CAN); and organic bases such as methylamine, ethylamine, diethylamine, triethylamine, piperidine; hydrogenating agents such as Pd/C, Pd (OH) 2/C (Pearlman's catalyst), palladium acetate, platinum oxide, platinum black, sodium borohydride, Na-liquid ammonia, Raney-Ni, Zn-acetic acid, tri (C₁-C₆)alkylsilanes, tri(C₁-C₆)alkylsilyl halides and the like.

According to one embodiment, the present invention provides a process for the preparation of ruxolitinib or a salt thereof, comprising:
reacting compound of formula 4 or salt thereof,

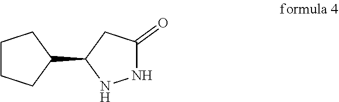 [SMILES: O=C1C[C@H](C2CCCC2)NN1]

with a compound formula 5.

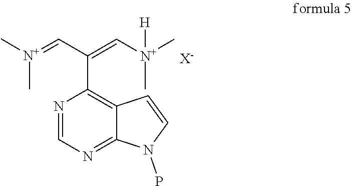

wherein, ‘P’ represents hydrogen or amine protecting group and X⁻ is counter anion.

In the aforementioned process, the compound of formula 4 can be a free base or a salt thereof and the salt of formula 4 can be a chiral salt or an achiral salt.

In the present invention, a chiral acid used to form the chiral salt may be selected from the following acids or an enantiomeric excess form thereof: mandelic acid, 2-chloromandelic acid, camphoric acid, lactic acid, malic acid, 3-bromocamphor-8-sulfonic acid, 3-bromocamphor-10-sulfonic acid, 10-camphorsulfonic acid, 2-amino-7,7-dimethylbicyclo[2,2, 1] heptan-1-methylene sulfonic acid, 2-acrylamide-7,7-dimethylbicyclo[2,2,1] heptan-1-methylene sulfonic acid, or tartaric acid and acyl derivatives thereof, preferably lactic acid, malic acid, camphoric acid, 10-camphorsulfonic acid, tartaric acid, diacetyl tartaric acid, dibenzoyl tartaric acid, di-p-toluoyl tartaric acid, di-p-anisoyl tartaric acid, di-p-chlorobenzoyl tartaric acid, di-p-bromobenzoyl tartaric acid, di-p-fluorobenzoyl tartaric acid, di-p-nitrobenzoyl tartaric acid, di-p-aminobenzoyl tartaric acid or di-p-cyanobenzoyl tartaric acid and the like; preferably tartaric acid, diacetyl tartaric acid, dibenzoyl tartaric acid or di-p-toluoyl tartaric acid.

In the present invention, a chiral acid used to form the chiral salt may be selected from D-tartaric acid, D-diacetyl tartaric acid, D-dibenzoyl tartaric acid, D-di-p-toluoyl tartaric acid, D-di-p-anisoyl tartaric acid, D-di-p-chlorobenzoyl tartaric acid, D-di-p-bromobenzoyl tartaric acid, D-di-p-fluorobenzoyl tartaric acid, D-di-p-nitrobenzoyl tartaric acid, D-di-p-aminobenzoyl tartaric acid, D-di-p-cyanobenzoyl tartaric acid and the like; preferably D-tartaric acid, D-dibenzoyl tartaric acid or D-di-p-toluoyl tartaric acid; more preferably D-tartaric acid.

In the present invention, the achiral salt is selected from hydrochloride, hydrobromide, nitrate, sulfate, phosphate, formate, acetate, trifluoroacetate, fumarate, oxalate, maleate, citrate, succinate, methanesulfonate, benzenesulfonate and p-toluene sulfonate, preferably hydrochloride, hydrobromide, sulfate, formate, acetate, trifluoroacetate, fumarate, maleate, methane sulphonate, or p-toluene sulphonate and the like; preferably hydrochloride or acetate.

In the aforementioned process, the X⁻ is a counter anion selected from but not limited to Cl⁻, Br⁻, I⁻, OMs⁻, OTf⁻, BF4⁻, PF6⁻, AsF6⁻, SbF6⁻, ClO4⁻ and the like; preferably Cl⁻.

The aforementioned process can be carried out in the presence of a suitable solvent; preferably polar-aprotic solvents, nitrile solvents, alcohol solvents, water or mixtures thereof; more preferably dimethylformamide, acetonitrile, ethanol, water or mixtures thereof.

In another embodiment, the present invention provides a process for the preparation of ruxolitinib or a salt thereof, comprising:
A) reacting compound formula 4 or salt thereof,

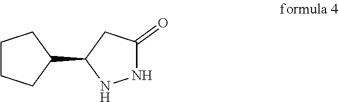 [SMILES: O=C1C[C@H](C2CCCC2)NN1]

with a compound formula 5,

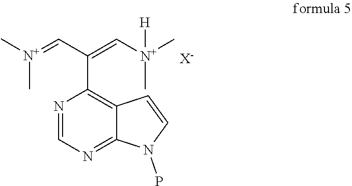

wherein, ‘P’ represents hydrogen or amine protecting group, X⁻ is counter anion; to provide acid intermediate of formula 6;

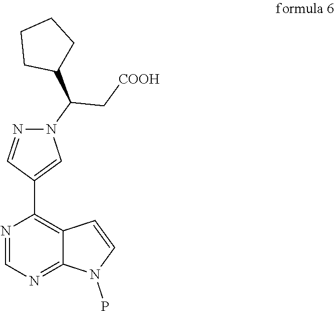

B) reacting the acid intermediate of formula 6 with alkyl chloroformates, dialkyl dicarbonates, alkyl or aryl sulphonyl chloride, sulfonic anhydrides to provide a compound of formula 7a;

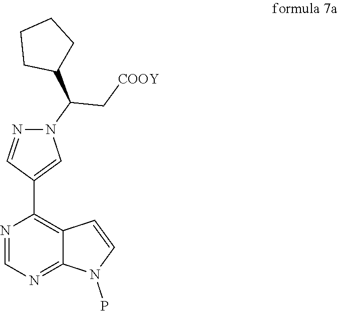

wherein ‘Y’ represents COOR, SO₂R and R is selected from alkyl, aryl or aralkyl group;
C) reacting the compound of formula 7a with an aminating agent to provide a compound of formula 8; and

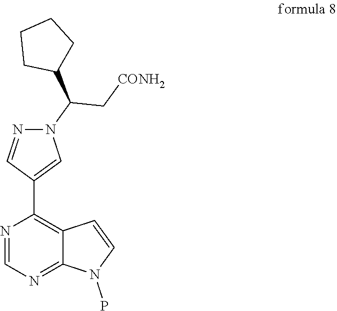

E) converting the compound of formula 8 into ruxolitinib or a salt thereof.

In step A) of the aforementioned process, the compound of formula 4 used can be a free base or a salt thereof and the salt of formula 4 can be a chiral salt or an achiral salt.

In the present invention, a chiral acid used to form the chiral salt may be selected from the following acids or an enantiomeric excess form thereof: mandelic acid, 2-chloromandelic acid, camphoric acid, lactic acid, malic acid, 3-bromocamphor-8-sulfonic acid, 3-bromocamphor-10-sulfonic acid, 10-camphorsulfonic acid, 2-amino-7,7-dimethylbicyclo[2,2,1] heptan-1-methylene sulfonic acid, 2-acrylamide-7,7-dimethylbicyclo[2,2,1] heptan-1-methylene sulfonic acid, or tartaric acid and acyl derivatives thereof, preferably lactic acid, malic acid, camphoric acid, 10-camphorsulfonic acid, tartaric acid, diacetyl tartaric acid, dibenzoyl tartaric acid, di-p-toluoyl tartaric acid, di-p-anisoyl tartaric acid, di-p-chlorobenzoyl tartaric acid, di-p-bromobenzoyl tartaric acid, di-p-fluorobenzoyl tartaric acid, di-p-nitrobenzoyl tartaric acid, di-p-aminobenzoyl tartaric acid or di-p-cyanobenzoyl tartaric acid and the like; preferably tartaric acid, diacetyl tartaric acid, dibenzoyl tartaric acid or di-p-toluoyl tartaric acid.

In the present invention, a chiral acid used to form the chiral salt may be selected from D-tartaric acid, D-diacetyl tartaric acid, D-dibenzoyl tartaric acid, D-di-p-toluoyl tartaric acid, D-di-p-anisoyl tartaric acid, D-di-p-chlorobenzoyl tartaric acid, D-di-p-bromobenzoyl tartaric acid, D-di-p-fluorobenzoyl tartaric acid, D-di-p-nitrobenzoyl tartaric acid, D-di-p-aminobenzoyl tartaric acid, D-di-p-cyanobenzoyl tartaric acid and the like; preferably D-tartaric acid, D-dibenzoyl tartaric acid or D-di-p-toluoyl tartaric acid; more preferably D-tartaric acid.

In the present invention, the achiral salt is selected from hydrochloride, hydrobromide, nitrate, sulfate, phosphate, formate, acetate, trifluoroacetate, fumarate, oxalate, maleate, citrate, succinate, methanesulfonate, benzenesulfonate and p-toluene sulfonate, preferably hydrochloride, hydrobromide, sulfate, formate, acetate, trifluoroacetate, fumarate, maleate, methane sulphonate, or p-toluene sulphonate and the like; preferably hydrochloride or acetate.

In step A) of the aforementioned process, the X⁻ is a counter anion selected from but not limited to Cl⁻, Br⁻, I⁻, OMs⁻, OTf⁻, BF₄⁻, PF₆⁻, AsF₆⁻, SbF₆⁻, ClO₄⁻ and the like; preferably Cl⁻.

The step A) reaction of the aforementioned process can be carried out in the presence of a suitable solvent; preferably polar-aprotic solvents, nitrile solvents, alcohol solvents, water or mixtures thereof; more preferably dimethylformamide, acetonitrile, ethanol water or mixtures thereof.

In step B) of the aforementioned process, the alkyl chloroformates, dialkyl dicarbonates, alkyl or aryl sulphonyl chloride, sulfonic anhydrides used is selected from ethyl chloroformate, methyl chloroformate, isobutyl chloroformate, dimethyl dicarbonate, diethyl dicarbonate, di-tert-butyl dicarbonate, methane sulfonyl chloride, ethane sulfonyl chloride, toluene sulfonyl chloride, methane sulfonic anhydride, toluene sulfonic anhydride and the like; preferably ethyl chloroformate or methane sulfonyl chloride; more preferably ethyl chloroformate.

The step B) reaction of the aforementioned process can be carried out in the presence of a base in a suitable solvent; wherein the base is selected from triethylamine, methylamine, ethylamine, 1,8-diazabicycle [5.4.0] undec7-ene (DBU); preferably triethylamine, and the suitable solvent is selected from chloro solvents such as dichloromethane, dichloroethane, chloroform, carbon tetrachloride and the like, or mixture thereof; preferably dichloromethane.

The intermediate compound of formula 7a obtained in step B) of the present invention can be proceed as in-situ or optionally isolated.

In step C) of the aforementioned process, the aminating agent used is selected from aqueous ammonia, liquid ammonia, ammonia gas and the like; preferably aqueous ammonia.

In step C) of the aforementioned process, the reaction can be carried out in the presence or absence of a suitable solvent.

In step D) of the aforementioned process, the conversion of the compound of formula 8 into ruxolitinib or a salt thereof can be carried out in the presence of a suitable dehydrating agent in a suitable solvent.

The suitable dehydrating agent is selected from phosphorus oxychloride, cyanuric chloride, phosphorus pentoxide, thionyl chloride, trifluoroacetic anhydride, trifluoro sulfonic anhydride, oxalyl chloride and the like; preferably phosphorus oxychloride, trifluoroacetic anhydride or cyanuric chloride; more preferably phosphorus oxychloride.

The suitable solvent is selected from chloro solvents such as dichloromethane, dichloroethane, chloroform, carbon tetrachloride and the like, polar-aprotic solvents such as dimethylacetamide, dimethylformamide, dimethyl sulfoxide, N-methylpyrrolidone and the like or mixture thereof; preferably dichloromethane or N-methylpyrrolidone or mixture thereof.

In step A) to step D) of the aforementioned process, the removing of protecting group (when “P” is amino protecting group) at any stage can be carried out in the presence of a suitable deprotecting agent.

The deprotecting agent used under an acidic condition is selected from trifluoroacetic acid, trifluoroacetic anhydride, lithium tetrafluoroborate, boron trifluoride-diethyl etherate and the like, preferably trifluoroacetic acid or boron trifluoride-diethyl etherate.

The deprotecting agent used under a basic condition is selected from aqueous or alcoholic solution of alkali metal hydroxides or alkaline earth metal hydroxide such as sodium hydroxide, potassium hydroxide, lithium hydroxide, magnesium hydroxide, barium hydroxide, calcium hydroxide, beryllium hydroxide, strontium hydroxide, radium hydroxide and the like, preferably aqueous sodium hydroxide or methanolic sodium hydroxide.

In another embodiment, the present invention provides a process for the purification of compound of formula 6, comprising:

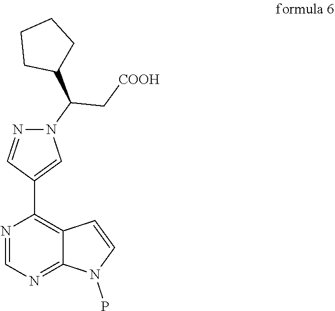

wherein, ‘P’ represents hydrogen or amine protecting group;
   reacting compound of formula 6 with dicyclohexylamine (DCHA) salt in a suitable solvent to provide dicyclohexylamine salt of compound of formula 6a, and

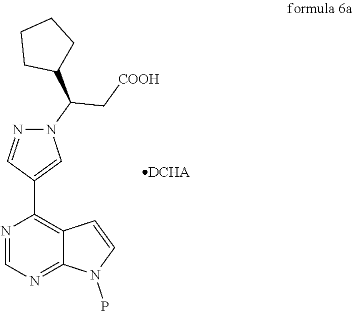

further converting dicyclohexylamine salt of compound of formula 6a into its free base by reacting it with base to provide pure compound of formula 6.

In the aforementioned process, the base used is selected from alkali metal hydroxides or alkaline earth metal hydroxide such as sodium hydroxide, potassium hydroxide, lithium hydroxide, magnesium hydroxide, barium hydroxide, calcium hydroxide, beryllium hydroxide, strontium hydroxide, radium hydroxide and the like; preferably sodium hydroxide; and the suitable solvent used is selected from ketone solvents such as acetone, methyl isobutyl ketone, methyl ethyl ketone and the like; preferably acetone.

In another embodiment, the present invention provides use of dicyclohexylamine salt of compound of formula 6a or pure compound of formula 6, in the preparation of ruxolitinib or a salt thereof.

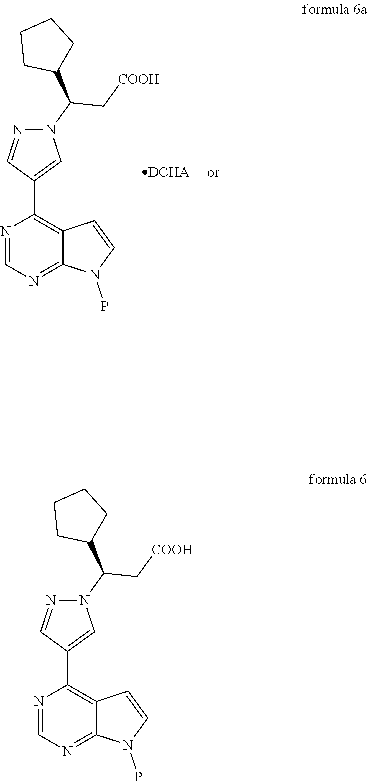

wherein, ‘P’ represents hydrogen or amine protecting group;

In another embodiment, the present invention provides a process for the preparation of ruxolitinib or a salt thereof, comprising:
A) reacting the acid intermediate of formula 6,

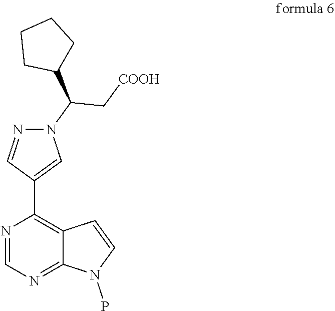

wherein, ‘P’ represents hydrogen or amine protecting group;
with alkyl chloroformates, dialkyl dicarbonates, alkyl or aryl sulphonyl chloride, sulfonic anhydrides to provide a compound of formula 7a;

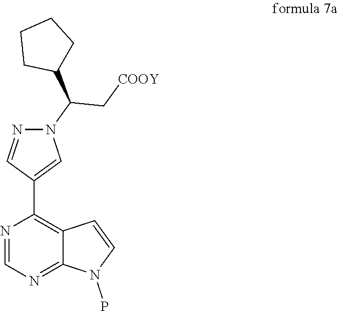

wherein ‘Y’ represents COOR, SO₂R and R is selected from alkyl, aryl or aralkyl group;
B) reacting the compound of formula 7a with an aminating agent to provide a compound of formula 8; and

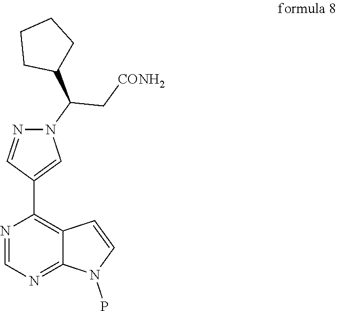

C) converting the compound of formula 8 into ruxolitinib or a salt thereof.

In another embodiment, the present invention provides a process for the preparation of ruxolitinib or a salt thereof, comprising:
A) reacting the acid intermediate of formula 6

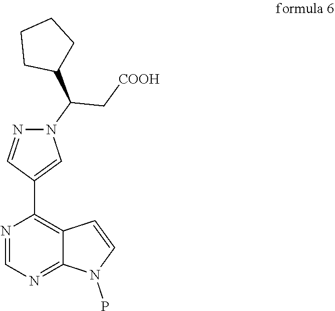

wherein, ‘P’ represents hydrogen or amine protecting group;
with coupling reagent, and followed by reaction with an aminating agent to provide a compound of formula 8; and

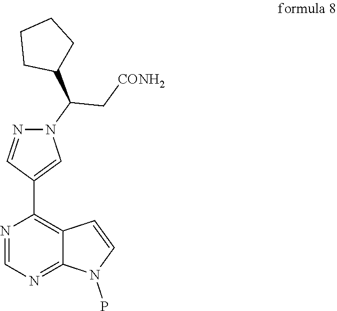

B) converting the compound of formula 8 into ruxolitinib or a salt thereof.

In the aforementioned process, the coupling reagent used is selected from 1,1′-carbonyldiimidazole (CDI), 1,1′-thiocarbonyldiimidazole, 1,3-dicyclohexylcarbodiimide, 1-ethyl-3-[3 (dimethylamino) propyl] carbodiimide hydrochloride and the like; preferably carbonyldiimidazole; and the aminating agent used is selected from aqueous ammonia, liquid ammonia, ammonia gas and the like; preferably aqueous ammonia.

In another embodiment, the present invention provides a process for the preparation of ruxolitinib or a salt thereof, comprising:
A) reacting compound of formula 4 or salt thereof,

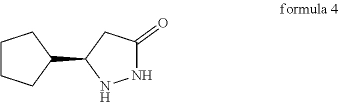 [SMILES: O=C1C[C@H](C2CCCC2)NN1]

with a compound formula 9,

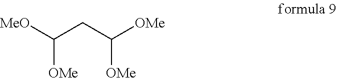 [SMILES: COC(CC(OC)OC)OC]

to provide a compound of formula 10;

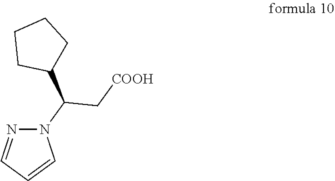 [SMILES: O=C(O)C[C@H](C1CCCC1)n1cccn1]

B) converting the compound of formula 10 into corresponding ester compound of general formula 11;

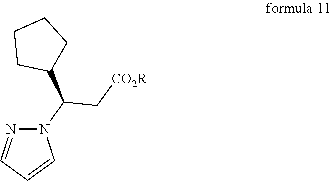

wherein ‘R’ represents C₁-C₄ alkyl group;
C) reacting the compound of general formula 11 with suitable halogenating agent to provide a compound of general formula 12;

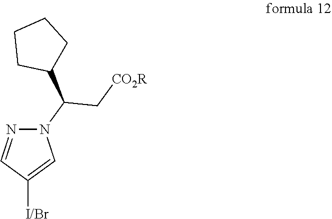

wherein ‘R’ defined above;
D) reacting the compound of general formula 12 with compound of general formula 13,

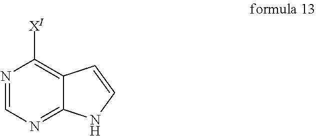

wherein, ‘X^{I}’ represents suitable leaving group;
to provide a compound of general formula 14;

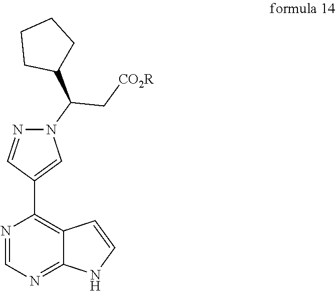

wherein ‘R’ defined above;
E) reacting the compound of general formula 14 with an aminating agent to provide a compound of formula 8; and

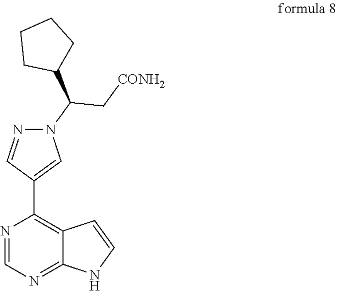

F) converting the compound of formula 8 into ruxolitinib or a salt thereof.

The following examples are provided as further detailed non-limiting illustrations of the technical solutions of the present application. They should not be construed as limiting the scope of the present application, but as merely illustrations and typical representatives of the present application.

### EXAMPLES

### Example 1

### Step 1: Preparation of 3-Cyclopentyl Acrylic Acid

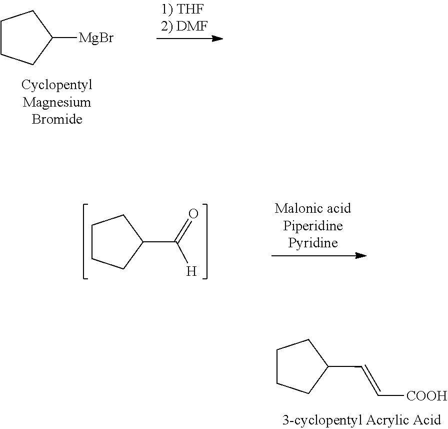

Charged THF (2 vol) and DMF (1.2 vol) into RBF and cooled to 0-5° C., add cyclopentyl Magnesium Bromide, 2.0 M in Ether (50.0 g, 1.0 eq.,) and raised the temperature to RT. Stirred the reaction mass at RT for 30-45 min (Monitor the progress of the reaction by TLC). After completion of the reaction, quenched the reaction by adding water (0.3 vol.) into the mass. Filtered the reaction mass, charged the filtrate into the RB flask and charged Pyridine (1.9 vol), Piperidine (0.01 vol) and Malonic acid (0.68 eq.) into RBF at RT and stirred for 5-10 min (Observation: Reaction mass is heterogeneous. Heated the reaction mixture to reflux and stirred for 7-8 hours under reflux. After completion of the reaction, cooled the reaction mass to 0-5° C., and adjusted the reaction mass pH to 1-2 by adding aq. 3N HCl (˜5.0 vol.,) at 0-5° C. Extracted the mass twice with Ethyl acetate (2×5 vol.,). Adjusted the pH of the organic layer to 11-12 by adding aq. 2M NaOH solution (˜20 vol.,) at 0-5° C. Separated the layers, wash the aq. Layer with Ethyl acetate (2×5 vol.,) and adjusted the pH of the aqueous layer with conc. HCl (˜3 vol.,) to 1-2 at 0-5° C. Further, extracted the compound with Ethyl acetate (2×7.5 vol.,) from aqueous layer, wash the organic layer with 5% aq. NaCl solution (2.5 vol.,) and distil off the solvent under reduced pressure to get the title compound.

Yield: ˜20 g

### Step 2: Preparation of 5-Cyclopentylpyrazolidin-3-one

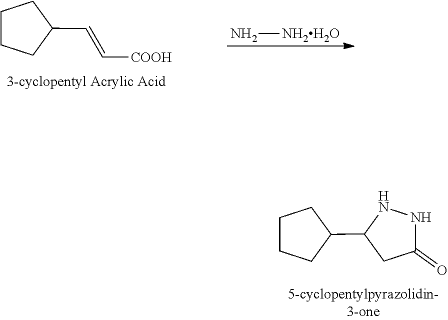

Charged 3-cyclopentyl Acrylic Acid (75.0 g, 1.0 eq.,) and hydrazine hydrate (75%, 1 vol.,) at RT. Heated the reaction mixture to 70-75° C. and stirred for 30-45 minutes at 70-75° C. After completion of the reaction, concentrated the reaction mass under reduced pressure at 70° C. to minimum volume, then charged water (1 vol.) into the reaction mixture, cooled the reaction mass to 0-5° C. and stirred for 12 hrs. Filtered the solid, washed with MTBE (0.5 vol., 37.5 mL) to give wet material (˜25 gm). Dried the compound at 45° C. to give 5-cyclopentylpyrazolidin-3-one (˜18 g, 1^{st} crop). Extracted the Filter mL's with DCM (˜5 vol.,), then distilled off the solvent under reduced pressure to get the title compound (˜17 gm as 2^{nd} crop).

Total weight=˜35 gm (1^{st}+2^{nd} crop).

### Step 3: Preparation of (R)-5-Cyclopentylpyrazolidin-3-one D-tartrate

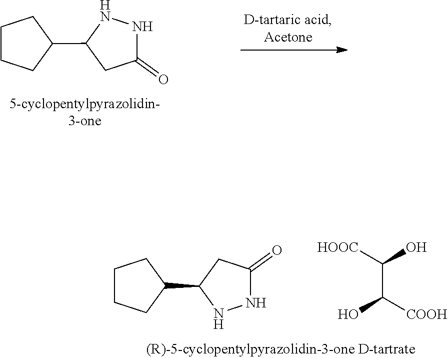 [SMILES: O=C1CC(C2CCCC2)NN1 ; O=C1C[C@H](C2CCCC2)NN1 ; O=C(O)[C@@H](O)[C@H](O)C(=O)O]

Charged 5-cyclopentylpyrazolidin-3-one (35.0 g, 1.0 eq.,) and acetone (10.0 vol.,) into RBF and stirred the mixture for 10-15 minutes at RT (Observation: Reaction mass is homogeneous). Charged D-(−)-tartaric acid (0.50 eq.,) into the reaction mixture, Stirred the heterogeneous mixture at RT for 30-45 minutes. Cooled the reaction mixture to 0-5° C. and stir for 1 hour at same temperature. Filtered the solid and washed with pre-cooled acetone (1.0 vol.,) to get wet material. Dried the compound under vacuum at 45-50° C. to get the title compound.

Yield: ˜30 g

### Example 2

### Step 1: Preparation of 4-Methyl-7H-pyrrolo[2,3-d]pyrimidine

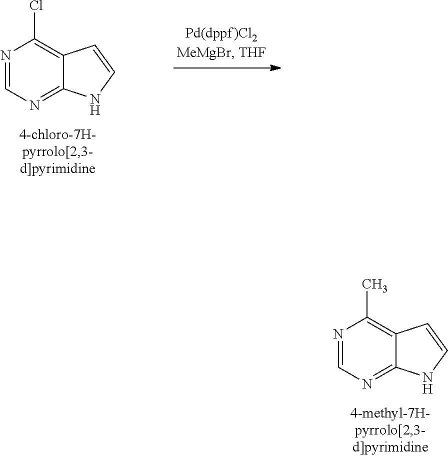

Charged 4-chloro-7H-pyrrolo[2,3-d]pyrimidine (SM, 25.0 g, 1.0 eq.,) and Pd (dppf)Cl₂·DCM (0.0071 eq.) and THF (5 vol.,) into RBF at RT and stirred for 30-45 min at same temperature. Cooled the mass to 0-5° C., added MeMgBr (3.0 M in diethyl ether, 2.8 eq.) into the reaction mass. Heated the reaction mass to 55-65° C. and stirred for 6-7 hours at same temperature (Monitor the progress of the reaction by TLC). After completion of the reaction, cooled the reaction mass to 0-5° C. and quenched with conc. HCl (˜3 vol.,) at 0-5° C. Water (˜4 vol.) was charged into reaction mixture and separated the layers. Adjusted the pH of the aq. layer to ˜7 by adding aq. NaHCO₃ solution (4 vol.,) and extracted with Ethyl acetate (3×6 vol.,). Treated the organic layer with activated charcoal (5.0%, 0.05 w/w, 1.25 g), distilled off solvent from the filtrate under reduced pressure to get crude compound. Further the crude compound was purified by silica column to get the title compound.

Yield: ˜10 g

### Step 2: Preparation of (E)-N-(3-(Dimethylamino)-2-(7H-pyrrolo[2,3-d] pyrimidin-4-yl) allylidene)-N-methylmethanaminium chloride hydrochloride

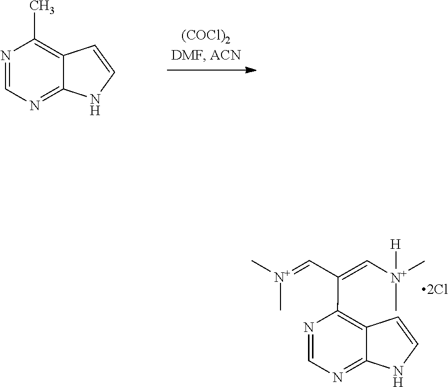

Charged acetonitrile (6.5 vol.,) and oxalyl chloride (2.0 eq.,) into RBF at RT. Cooled the reaction mass to 0-5° C. and added DMF (7.5 vol.,). Raised the reaction mass temperature to RT and charged Step-1 compound (13.0 g, 1.0 eq.,) into the reaction mixture and heated the reaction mixture to 80-85° C. and stirred for 6 h at 80-85° C. After completion of the reaction, charged THF (20 vol.) at 40-45° C., and maintained the reaction mass at RT for 12 h. Settled the reaction mass for 30-45 min, remove upper THF layer, washed twice with MTBE (2×5 vol.,), filtered the reaction mass under Nitrogen/inert atmosphere (hygroscopic nature) to get the title compound.

Yield: ˜5 g

### Example 3

### Preparation of (R)-3-(4-(7H-Pyrrolo[2,3-d]pyrimidin-4-yl)-1H-pyrazol-1-yl)-3-cyclopentylpropanoic acid

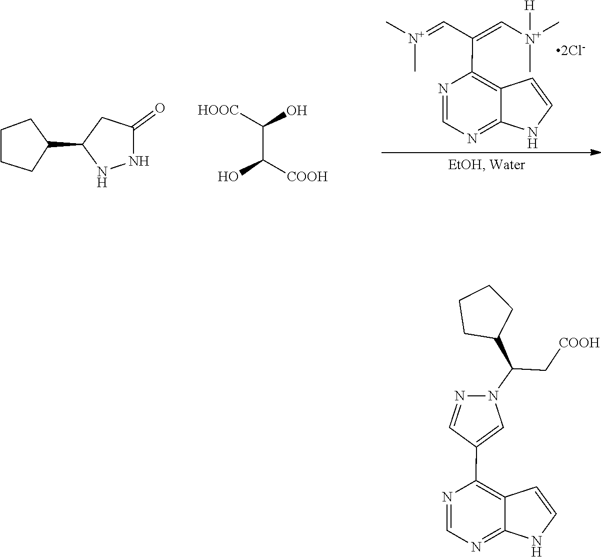

Charged (R)-5-cyclopentylpyrazolidin-3-one D-tartrate (30.0 g, 1.0 eq.,), Ethanol (6 vol.,) and water (3 vol.,) into RBF at RT. Then charged (E)-N-(3-(Dimethylamino)-2-(7H-pyrrolo[2,3-d]pyrimidin-4-yl) allylidene)-N-methylmethanaminium chloride hydrochloride (1.1 eq.,) and heated the reaction mixture at 70-75° C. for 6-7 h. After completion of the reaction, distilled off the solvent under reduced pressure at below 50° C., then adjusted the pH to ˜7-8 by adding sat. aq. NaHCO₃ solution (20.0 vol.,) at RT. Washed the reaction mass with DCM (3×6 vol.,), then adjusted pH of the aq. layer to ˜1-2 by adding conc. HCl (3.0 vol.,) at 0-5° C. Extracted the compound with ethyl acetate (3×12 vol.), then distil off organic layer under reduced pressure to get the title compound.

Yield: 13.0 g

### Example 4

### Step 1: Preparation of Benzyl 4-methyl-7H-pyrrolo[2,3-d]pyrimidine-7-carboxylate

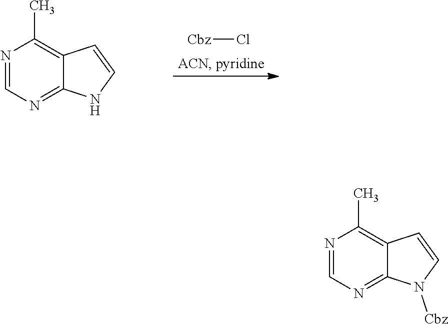

Charged 4-Methyl-7H-pyrrolo[2,3-d]pyrimidine (100 g, 1.0 eq.,), acetonitrile (3 V) into RBF at 25-35° C. under nitrogen atmosphere. Pyridine (5 V) was added into the mass and stirred for 10-15 minutes at same temperature. The reaction mass was cooled to 0 to −5° C., add benzyl chloroformate (2.0 eq.,) was added at below 10° C., temperature was raised to 25-35° C. and stirred for 6-8 hours at same temperature. After completion of the reaction, the reaction mass was cooled to 0-10° C., water (25 V) was added at 0 to −5° C. and stirred for 1-2 hours at 0 to −5° C. The solid obtained was filtered, washed with aq. ACN (1:1 ratio, 2×2 V) and dried under vacuum at 50-60° C. to get the title compound.

Yield: ˜160 g

### Step 2: Preparation of (R)-3-(4-(7H-pyrrolo[2,3-d]pyrimidin-4-yl)-1H-pyrazol-1-yl)-3-cyclopentylpropanoic acid

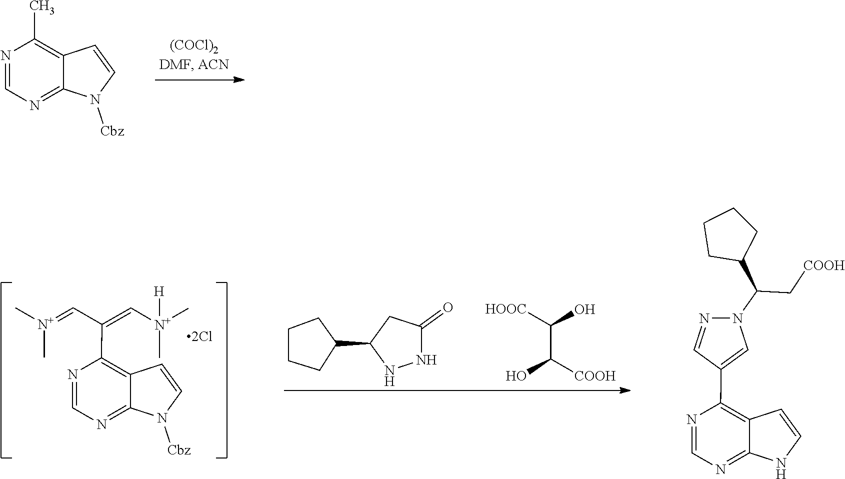

Charged acetonitrile (6.5 V) into RBF at 20-30° C. under nitrogen atmosphere and cooled to 0-10° C., add oxalyl chloride (2.1 eq.,) and stirred for 10-20 min, then slowly add DMF (15.0 eq.,) and stirred for 1-2 hours at same temperature. Then the temperature of the reaction mass was raised to 20-30° C., was added Benzyl 4-methyl-7H-pyrrolo[2,3-d]pyrimidine-7-carboxylate (90 g) and stirred for 10-15 min at RT. Again the temperature of the reaction mass was heated to 70-80° C. and stirred for 6-8 hours at same temperature. After completion of the reaction, charged THF (20 V) into the reaction mass and stirred overnight. The organic layer was decanted and the mass was washed with MTBE (5×5 V) and dried the compound under vacuum to get vilsmeier salt (91 g). To the Vilsmeier salt, ethanol (6.0 V) and water (3.0 V) were charged at RT, stirred for 5-10 min and add (R)-5-cyclopentylpyrazolidin-3-one D-tartrate (1.0 eq.,) was added at same temperature. The reaction mass was heated to 70-80° C., stirred for 6-8 hours at same temperature and cooled to 20-30° C. Distilled-off the solvent under vacuum and added aq. HCl (3.0 V), heated to 70-80° C. and stirred for 1-2 hours at same temperature. The reaction mass was cooled to 0-5° C., pH of the mass was adjusted to ˜8 with aq. NaHCO₃ solution (20 v) and washed with Ethyl acetate (12 V). The pH of the reaction mass adjusted to ˜3 by adding aq. HCl solution (3.0 V) at 0-5° C. The compound of reaction mass extracted with ethyl acetate (2×12 V) and distilled off the solvent under reduced pressure to get the title compound.

Yield: 37 g

### Step 3: Purification of (R)-3-(4-(7H-pyrrolo[2,3-d]pyrimidin-4-yl)-1H-pyrazol-1-yl)-3-cyclopentylpropanoic acid

Charged crude (R)-3-(4-(7H-pyrrolo[2,3-d]pyrimidin-4-yl)-1H-pyrazol-1-yl)-3-cyclopentylpropanoic acid (328.0 g, 1.0 eq.,) and acetone (10 V) into RBF at RT. The reaction mass heated to 40-50° C., charge activated carbon (0.05 T) and maintained for 30-45 min at same temperature. The reaction mass was filtered on celite bed (0.2 T) and washed with acetone (1 V). The obtained filtrate was taken into RBF, heated to 40-50° C. and add Dicyclohexylamine (DCHA, 1.0 to 1.5 eq., until the pH of the mass is 9 to 9.5) and maintained for 1-2 hours at 50-60° C. Then the reaction mass was cooled to RT and stirred for 1-2 hours at same temperature. The solid obtained was filtered and washed with acetone (1.0 V) to obtain wet compound. The obtained wet compound (230 g) and acetone (5 V) were charged into RBF at RT, heated to 50-60° C. and maintained for 1-2 hours at same temperature. The reaction mass was cooled to RT and stirred for 1-2 hours at same temperature. The solid obtained was filtered, washed with acetone (1 V) and dried in VTD for 6-8 hours (198.0 g) to obtain DCHA salt.

To the above obtained DCHA salt, charge water (5 V) and adjusted the reaction mass pH to 13-14.0 by adding aq. Sodium hydroxide solution. The aqueous layer was washed with MTBE (3×6 V) and adjusted the aqueous layer pH to 5.2 to 5.5 by adding aq. HCl solution. The reaction mass was extracted with 10% methanol in ethyl acetate (4×5 V) and the organic layer was washed with sat. aq. NaCl solution (2 V). To the organic layer, charged charcoal (0.1 T) at 40-50° C., stirred for 1-2 h, filtered on celite bed and distilled-off the filtrate under reduced pressure at below 55° C. until 4.0-5.0 vol. n-Heptane (5 V) was added to the reaction mass at 40-50° C., gradually cooled the mass to RT and stirred for 3-4 hours at same temperature. The solid obtained was filtered, washed with n-heptane (1 V) and dried under VTD at 50-55° C. for 6-8 hours to get the pure title compound.

Yield: 103 g

### Step 4: Preparation of (R)-3-(4-(7H-pyrrolo[2,3-d]pyrimidin-4-yl)-1H-pyrazol-1-yl)-3-Cyclopentylpropionamide

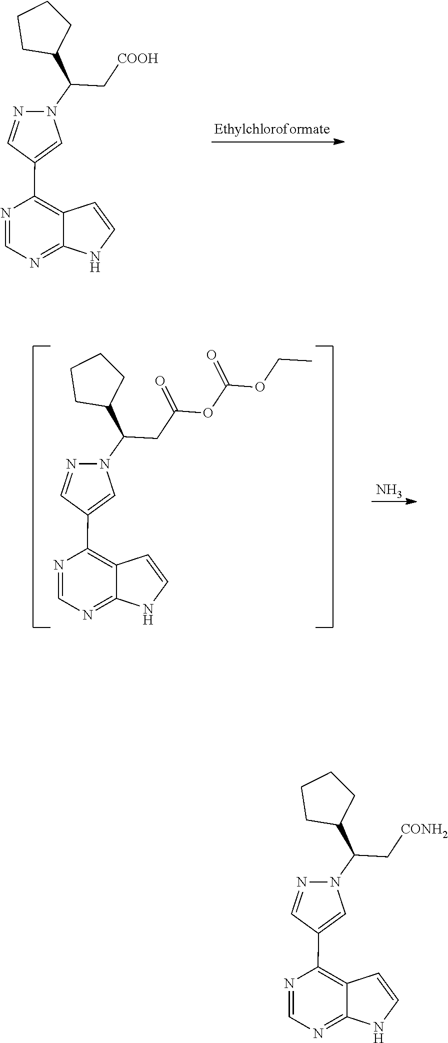

Charged (R)-3-(4-(7H-pyrrolo[2,3-d]pyrimidin-4-yl)-1H-pyrazol-1-yl)-3-cyclopentylpropanoic acid (crude, 45.0 g, 1.0 eq.,), DCM (10.0 V.), TEA (1.3 eq.) into RBF at RT and cooled to-15 to −25° C., then added ethyl chloroformate (1.10 eq.) and maintained for 1-2 hours at same temperature (Monitor the progress of the reaction by TLC). After completion of the reaction, the reaction mass was quenched with methanolic ammonia (˜10 V.,) and maintained 1-2 hours at same temperature. Then the temperature of the reaction mass was raised to RT, water (10 v) was charged and stirred for 20-30 min. Separated the layers, the aqueous layer was extracted with DCM (4×10 V) at RT. Combine organic layers, wash with sat. NaHCO₃ solution (10 v) and distilled off the solvent under reduced pressure to get the title compound.

Yield: 35 g

### Step 5: Preparation of Ruxolitinib

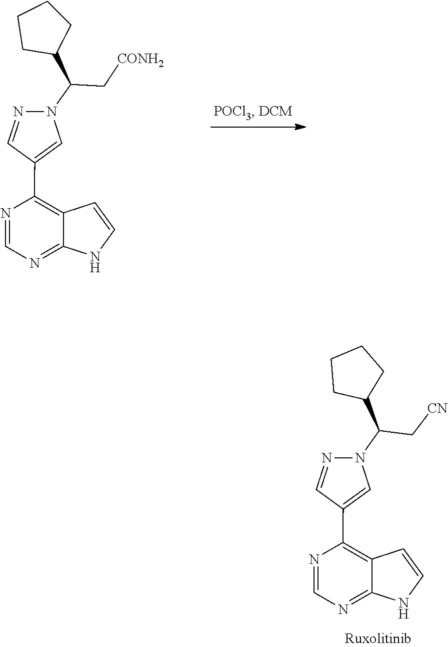 [SMILES: NC(=O)C[C@H](C1CCCC1)n1cc(-c2ncnc3[nH]ccc23)cn1 ; N#CC[C@H](C1CCCC1)n1cc(-c2ncnc3[nH]ccc23)cn1]

Charged (R)-3-(4-(7H-pyrrolo[2,3-d]pyrimidin-4-yl)-1H-pyrazol-1-yl)-3-cyclopentylpropionamide (5.0 g, 1.0 eq.,), DCM (20.0 V.,) and NMP (2.0 V.,) into the RBF at RT under nitrogen atmosphere. The reaction mass was cooled to below 10° C., then POCl₃ (4.1 eq.,) was added into the reaction mass and maintained for an hour at 20-30° C. After completion of the reaction, the temperature of the reaction mass raised to RT, adjusted the pH to ˜7.0 by adding aq. NaHCO₃ solution (˜50 V.,). Separated the layers and the aqueous layer extracted with DCM (3×10 V). The combined organic layer was washed with sat. NaCl solution (17 V) and distilled off the organic layer under reduced pressure to give crude compound (7.0 g). The obtained crude compound was purified by silica column to get the title compound.

Yield: 3 g

### Step 6: Preparation of Ruxolitinib Phosphate

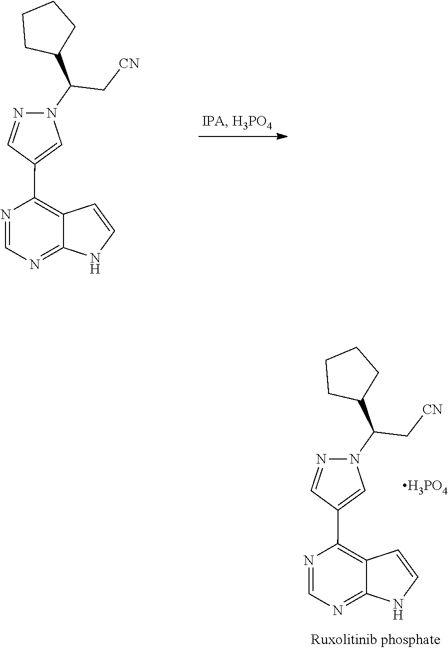

Charged Ruxolitinib (11.0 g, 1.0 eq.,) and IPA (37 V) into RBF at RT and stirred for 30 min. Ortho phosphoric acid solution (1.1 eq., of ortho phosphoric acid in 3 V IPA) was added into the reaction mass at RT and stirred for 1 hour under reflux. Then cooled the reaction mass to RT and stirred for 2 hours at RT. The solid obtained was filtered, washed with IPA (4 V) and dried under vacuum at 40-50° C. for 6-8 hours to get crude compound (11.60 g). The obtained crude compound was charged with IPA (30 V), heated to 50-60° C. and maintained for 2-3 hours at same temperature. The reaction mass was filtered at 30-40° C., washed with IPA (5 V) and dried under reduced pressure to get the title compound.

Yield: 8.3 g

1. A process for the preparation of ruxolitinib or a salt thereof, comprising:
A) reacting compound formula 4 or salt thereof, 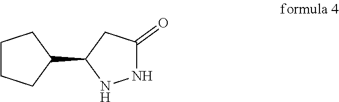 [SMILES: O=C1C[C@H](C2CCCC2)NN1]
with a compound formula 5, 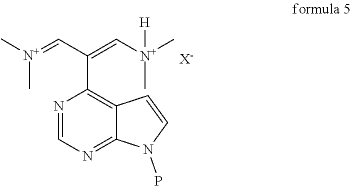
wherein, ‘P’ represents hydrogen or amine protecting group, and X⁻ is counter anion;
to provide acid intermediate of formula 6; 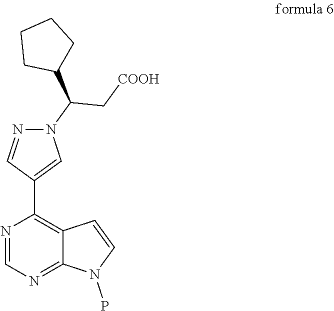
B) optionally, purifying the acid intermediate of formula 6 by reacting it with dicyclohexylamine salt in a suitable solvent to provide dicyclohexylamine salt of acid intermediate of formula 6a, and 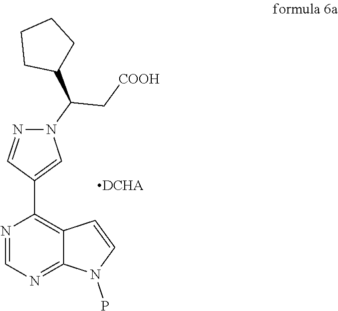
further converting into its free base by reacting it with base to provide pure acid intermediate of formula 6;
C) reacting the acid intermediate of formula 6 with alkyl chloroformates, dialkyl dicarbonates, alkyl or aryl sulphonyl chloride, sulfonic anhydride to provide a compound of formula 7a; 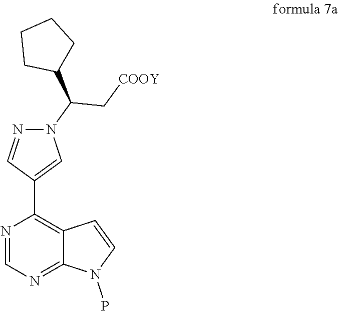
wherein ‘Y’ represents COOR, SO₂R and R is selected from alkyl, aryl or aralkyl group;
D) reacting the compound of formula 7a with an aminating agent to provide a compound of formula 8; and 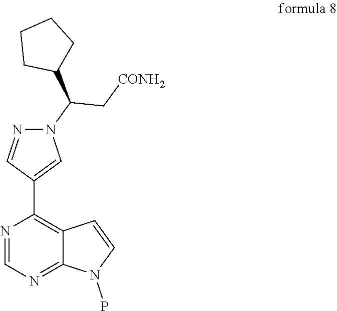
E) converting the compound of formula 8 into ruxolitinib or a salt thereof.

2. The process as claimed in claim 1, wherein the amine protecting group is selected from benzyloxycarbonyl, 2,2,2-trichloroethoxycarbonyl, 2-(trimethylsilyl) ethoxycarbonyl, 2-(4-trifluoromethylphenylsulfonyl) ethoxycarbonyl, tert-butoxycarbonyl, 1-adamantyloxycarbonyl, 2-adamantylcarbonyl, 2,4-dimethylpent-3-yloxycarbonyl, cyclohexyloxycarbonyl, 1,1-dimethyl-2,2,2-trichloroethoxycarbonyl, vinyl, 2-chloroethyl, 2-phenylsulfonylethyl, p-nitrophenylsulfonyl, p-toluenesulfonyl, phenylsulfonyl, methanesulfonyl, allyl, benzyl, 2-nitrobenzyl, 4-nitrobenzyl, diphenyl-4-pyridylmethyl, N′, N′-dimethylhydrazino, methoxymethyl, tert-butoxymethyl, benzyloxymethyl, 2-tetrahydropyranyl, tri (C1-4alkyl) silyl, 1,1-diethoxymethyl, 2-(trimethylsilyl) ethoxymethyl and N-pivaloyloxymethyl.

3. The process as claimed in claim 1, wherein the X⁻ is a counter anion selected from but not limited to Cl⁻, Br⁻, I⁻, OMs⁻, OTf⁻, BF₄⁻, PF₆⁻, AsF₆⁻, SbF₆⁻ and ClO₄⁻.

4. The process as claimed in claim 1, wherein the alkyl chloroformates, dialkyl dicarbonates, alkyl or aryl sulphonyl chloride, sulfonic anhydride selected from ethyl chloroformate, methyl chloroformate, isobutyl chloroformate, dimethyl dicarbonate, diethyl dicarbonate, di-tert-butyl dicarbonate, methane sulfonyl chloride, ethane sulfonyl chloride, toluene sulfonyl chloride, methane sulfonic anhydride and toluene sulfonic anhydride.

5. The process as claimed in claim 1, wherein the aminating agent is selected from aqueous ammonia, liquid ammonia and ammonia gas.

6. A process for the preparation of acid intermediate of formula 6, comprising:
reacting compound formula 4 or salt thereof, 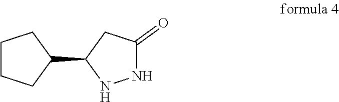 [SMILES: O=C1C[C@H](C2CCCC2)NN1]
with a compound formula 5, 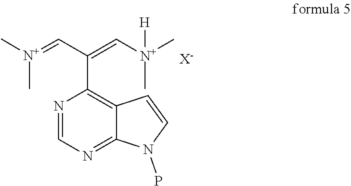
wherein, ‘P’ represents hydrogen or amine protecting group, and X-is counter anion;
to provide acid intermediate of formula 6.

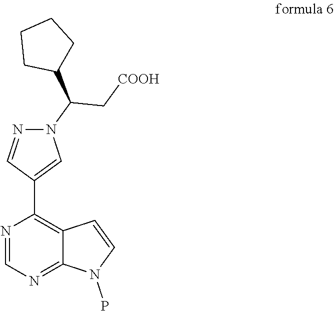

7. A process for the purification of compound of formula 6, comprising: 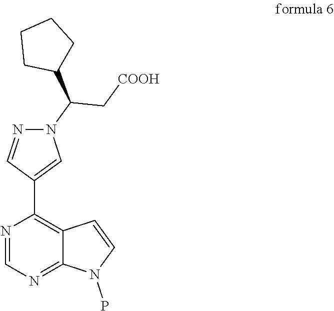
wherein, ‘P’ represents hydrogen or amine protecting group;
reacting compound of formula 6 with dicyclohexylamine salt in a suitable solvent to provide dicyclohexylamine salt of compound of formula 6a, and 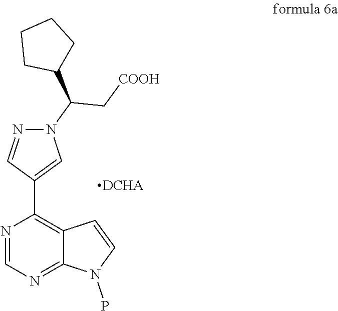
further converting dicyclohexylamine salt of compound of formula 6a into its free base by reacting it with base to provide pure compound of formula 6.

8. A process for the preparation of ruxolitinib or a salt thereof, comprising:
A) reacting the acid intermediate of formula 6, 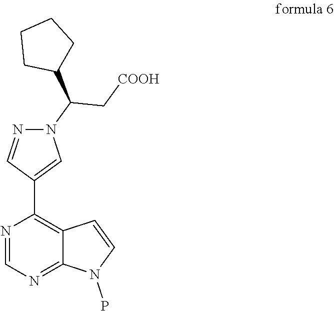
wherein, ‘P’ represents hydrogen or amine protecting group;
with alkyl chloroformates, dialkyl dicarbonates, alkyl or aryl sulphonyl chloride, sulfonic anhydride to provide a compound of formula 7a; 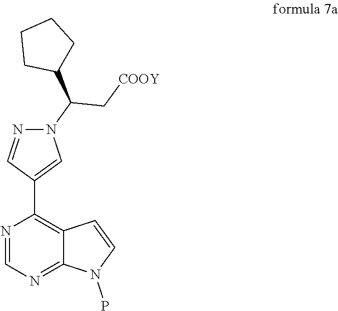
wherein ‘Y’ represents COOR, SO₂R and R is selected from alkyl, aryl or aralkyl group;
B) reacting the compound of formula 7a with an aminating agent to provide a compound of formula 8; and 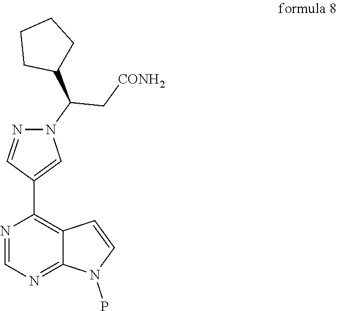
C) converting the compound of formula 8 into ruxolitinib or a salt thereof.

9. (canceled)

10. A compound of formula 7a: 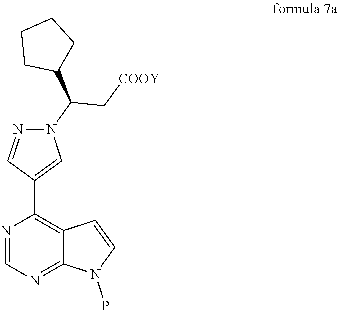
wherein, ‘P’ represents hydrogen or amine protecting group; and
‘Y’ represents COOR, SO2R and R is selected from alkyl, aryl or aralkyl group.
